SEQUENCE LISTING (1 sequence)

SEQ ID NO: 1 - protein, 1113 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEWMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

(19) United States
(12) Patent Application Publication
(10) Pub. No.: US 2023/0321098 A1
(43) Pub. Date: Oct. 12, 2023

(54) OSIMERTINIB AND SELPERCATINIB COMBINATIONS FOR THE TREATMENT OF EGFR- AND RET-ASSOCIATED CANCERS

(71) Applicant: Loxo Oncology, Inc., Indianapolis, IN (US)

(72) Inventors: Alexander E. DRILON, New York, NY (US); Helena A. YU, New York, NY (US)

(21) Appl. No.: 18/041,617

(22) PCT Filed: Aug. 25, 2021
(86) PCT No.: PCT/US2021/047496

§ 371 Date: Feb. 14, 2023

Related U.S. Application Data
(60) Provisional application No. 63/069,981, filed on Aug. 25, 2020.

(51) Int. Cl. A61K 31/506; A61K 31/4995

(52) U.S. Cl. CPC A61K 31/506; A61K 31/4995

(57) ABSTRACT

The present disclosure provides methods for treating patients with both EGFR- and RET-associated cancers with a combination of osimertinib and selpercatinib, in particular when treatment with the osimertinib leads to the development of fusions or mutations related to RET-associated cancers.

### TECHNICAL FIELD

This disclosure relates to methods of medical treatment, more particularly to the treatment of RET- and EGFR-associated cancers using a combination of selpercatinib and osimertinib.

### BACKGROUND

Rearranged during Transfection (RET) is a single-pass transmembrane receptor belonging to the tyrosine kinase superfamily that is required for normal development, maturation, and maintenance of several tissues and cell types. The extracellular portion of the RET kinase contains four calcium-dependent cadherin-like repeats involved in ligand binding and a juxtamembrane cysteine-rich region necessary for the correct folding of the RET extracellular domain, while the cytoplasmic portion of the receptor includes two tyrosine kinase subdomains.

RET signaling is mediated by the binding of a group of soluble proteins of the glial cell line-derived neurotrophic (GDNF) family ligands (GFLs), which also includes neurturin (NTRN), artemin (ARTN) and persephin (PSPN). Unlike other tyrosine kinases, RET does not directly bind to GFLs and requires an additional co-receptor: that is, one of four GDNF family receptor-α (GFRα) family members, which are tethered to the cell surface by a glycosylphosphatidylinositol linkage. GFLs and GFRα family members form binary complexes that in turn bind RET and recruit it into cholesterol-rich membrane subdomains, which are known as lipid rafts, where RET signaling occurs.

Upon binding of the ligand co-receptor complex, RET dimerizes and autophosphorylation on intracellular tyrosine residues occurs. These actions subsequently recruit adaptor and signaling proteins to stimulate multiple downstream pathways. Adaptor protein binding to these docking sites leads to activation of RAS-MAPK and PI3K-Akt/mTOR signaling pathways or to recruitment of the CBL family of ubiquitin ligases that functions in RET downregulation of RET-mediated functions.

Aberrant RET expression and/or activity has been demonstrated in different cancers. RET fusions are implicated in several cancers, including papillary thyroid carcinoma and non-small cell lung cancer. Genomics analyses have further identified RET fusions in breast and colon cancer patient samples, providing therapeutic rational for the use of RET inhibitors in multiple patient subpopulations. The identification of RET fusions in some cancers has prompted the use of RET inhibitors to treat patients whose tumors express a RET fusion protein. However, one of the greatest challenges in treating cancer is the ability of tumor cells to become resistant to therapy, particularly to tyrosine kinase inhibitor therapies such as RET inhibitors. Kinase reactivation by mutation is a common mechanism of resistance. When resistance occurs, the patient's treatment options are often very limited, and the cancer progresses unchecked in most circumstances.

Thus, there is a clear need for methods for the treatment of EGFR- and RET-associated cancers, particularly when treatment with an EGFR inhibitor leads to the development of fusions or mutations related to RET-associate cancers.

### SUMMARY

The present disclosure provides methods for treating patients with both an EGFR-associated cancer and a RET-associated cancer with a combination of selpercatinib and osimertinib. In particular, the administration of osimertinib and selpercatinib in combination may be used in the treatment of patients with EGFR-associated cancers being treated with osimertinib that subsequently develop fusions or mutations indicative of a RET-associated cancer.

Thus, in one aspect, a method of treating a patient with an EGFR-associated cancer and a RET-associated cancer, comprising administering to the patient a therapeutically effective amount of selpercatinib, or a pharmaceutically acceptable salt thereof, and a therapeutically effective amount of osimertinib, or a pharmaceutically acceptable salt thereof, is disclosed.

In another aspect, disclosed herein is a method of treating a patient with an EGFR-associated cancer and a RET-associated cancer, comprising administering to the patient a therapeutically effective amount of selpercatinib, or a pharmaceutically acceptable salt thereof, and a therapeutically effective amount of osimertinib, or a pharmaceutically acceptable salt thereof; wherein the RET-associated cancer is associated with a dysregulation of a RET gene, a RET kinase, or the expression or activity or level of any of the same; and wherein the dysregulation of a RET gene, a RET kinase, or the expression or activity or level of the same comprises a RET gene fusion.

In some embodiments, the RET-associated cancer is associated with a dysregulation of a RET gene, a RET kinase, or the expression or activity or level of any of the same. In some embodiments, the dysregulation of a RET gene, a RET kinase, or the expression or activity or level of the same comprises a RET gene fusion. In some embodiments, the dysregulation of a RET gene, a RET kinase, or the expression or activity or level of the same comprises one or more point mutations in the RET gene.

In some embodiments, the dysregulation of a RET gene, a RET kinase, or the expression or activity or level of any of the same comprises one or more RET inhibitor resistance mutations.

In a further aspect, a method of treating a RET-associated cancer in a patient in need thereof is provided, the method comprising: administering therapeutically effective amount of selpercatinib, or a pharmaceutically acceptable salt thereof, to the patient; determining if the patient has one or more EGFR-associated resistance mutations following administration of selpercatinib or its pharmaceutically acceptable salt; and if the patient is determined to have one or more EGFR-associated resistance mutations, administering to the patient a therapeutically effective amount of osimertinib, or a pharmaceutically acceptable salt thereof, and a therapeutically effective amount of selpercatinib, or a pharmaceutically acceptable salt thereof.

In some embodiments, osimertinib is administered as a mesylate salt, i.e., osimertinib mesylate. In some embodiments, osimertinib mesylate is administered orally once a day. In some embodiments, osimertinib mesylate is administered as a 40 mg dose or an 80 mg dose.

In some embodiments, selpercatinib is administered as a free base. In some embodiments, selpercatinib is administered orally twice a day. In some embodiments, selpercatinib is administered twice a day as a 40 mg dose, an 80 mg dose, a 120 mg dose, or a 160 mg dose.

In some embodiments, the RET-associated cancer is selected from the group consisting of: lung cancer, papillary thyroid cancer, medullary thyroid cancer, differentiated thyroid cancer, recurrent thyroid cancer, refractory differentiated thyroid cancer, multiple endocrine neoplasia type 2A or 2B, pheochomocytoma, parathyroid hyperplasia, breast cancer, colorectal cancer, papillary renal cell carcinoma, ganglioneuromatosis of the gastroenteric mucosa, and cervical cancer.

The details of one or more embodiments of the disclosure are set forth in the description below. Other features, objects, and advantages of the disclosure will be apparent from the description and from the claims.

### DETAILED DESCRIPTION

Many modifications and other embodiments disclosed herein will come to mind to one skilled in the art to which the disclosed methods pertain having the benefit of the teachings presented in the foregoing descriptions and the associated drawings. Therefore, it is to be understood that the disclosures are not to be limited to the specific embodiments disclosed and that modification and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from and combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, wherein a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plan meaning derived from grammatical organization or punctuation, or the number or type of aspects described in the specification.

All publications mentioned herein are incorporated by reference to disclose and describe the methods and/or materials used in the methods in connection with which the publications are cited. The publications discussed herein are provided solely for their disclosed prior to the filing date of the present application.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the present of the stated features, integers, steps, or components as referred to, but does not preclude the present or addition of one or more features, integers, steps, or components, or groups thereof. Moreover, each of the terms “by”, “comprising”, “comprises”, “comprised of”, “including”, “includes”, “included”, “involving”, “involves”, “involved”, and “such as” are used in their open, non-limiting sense and may be used interchangeably. Further, the term “comprising” is intended to include examples and aspects encompassed by the terms “consisting essentially of” and “consisting of”. Similarly, the term “consisting essentially of” is intended to include examples encompassed by the term “consisting of”.

As used in the specification and the appended claims, the singular forms “a”, “an” and “the” include plural referents unless the context clearly dictates otherwise. Thus, for example, reference to “a therapeutic agent” or “a clinical condition”, includes, but is not limited to, two or more such therapeutic agents or clinical conditions, and the like.

As used herein, the terms “treating”, “treatment”, or “to treat” includes restraining, slowing, stopping, or reversing the progression or severity of an existing symptom or disorder.

As used herein, the term “patient” refers to a mammal, in particular a human.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirably to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts of submultiples to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safest dose according to sound medical judgement. It will be understood by those of ordinary skill in the art, however, that a patient may insist upon a lower dose or tolerable dose for medical reasons, psychological reasons or for virtually any other reason.

A response to a therapeutically effective dose of a compound as used herein can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dosage administrations daily, for one or several days. Guidance can be found in the literature for appropriate dosages for given classes of pharmaceutical products.

As used herein, the term “optional” or “optionally” means that the subsequently described event or circumstance can or cannot occur, and that the description includes instances where said event or circumstance occurs and instances where it does not.

As used interchangeably herein, “subject”, “individual”, or “patient” can refer to a vertebrate organism, such as a mammal (e.g., a human). “Subject” can also refer to a cell, a population of cells, a tissue, an organ, or an organism, preferably to a human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein can include any treatment of a disorder in a patient, particularly a human, and can include any one or more of the following: a) preventing the disease from occurring in a patient which may be predisposed to the disease but has not yet been diagnosed as having it; b) inhibiting the disease, i.e., arresting its development; and (c) relieving the disease, i.e., mitigating or ameliorating the disease and/or its symptom or conditions. The term “treatment” as used herein can refer to therapeutic treatment alone, prophylactic treatment alone, or both therapeutic and prophylactic treatment. Those in need of treatment (i.e., patients in need thereof) can include those already with the disorder and/or those in which the disorder is to be prevented. As used herein, the term “treating”, can include inhibiting the disease, disorder, or condition, i.e., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a patient by administration of an analgesic agent even though such agent does not treat the cause of the pain.

As used herein, “dose”, “unit dose”, or “dosage” can refer to physically discrete units suitable for use in a patient, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing the rate of advancement of a disease, disorder, condition, or side effect.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by system reduction of the compound structure using naming conventions, or by commercially available software, such as CHEMDRAW™ (Cambridgesoft Corporation, U.S.A.).

In one aspect, the present disclosure provides methods for the treatment of a patient with an EGFR-associated cancer and a RET-associated cancer by administering a therapeutically effective amount of selpercatinib, or a pharmaceutically acceptable thereof, and a therapeutically effective amount of osimertinib, or a pharmaceutically acceptable salt thereof.

Selpercatinib, having the chemical name of 6-(2-hydroxy-2-methylpropoxy)-4-(6-(6-6-methoxypyridin-3-yl)methyl)-3,6-diazabicyclo[3.1.1]heptan-3-yl)pyridine-3-yl)pyrazolo[1,5-a]pyridine-3-carbonitrile, is RET inhibitor having the chemical structure:

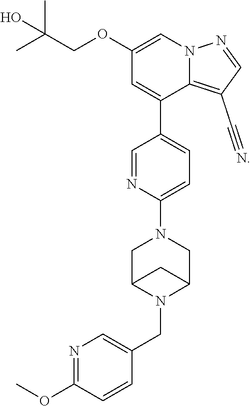 [SMILES: COc1ccc(CN2C3CC2CN(c2ccc(-c4cc(OCC(C)(C)O)cn5ncc(C#N)c45)cn2)C3)cn1]

In some embodiments, selpercatinib is administered as a free base. In some embodiments, selpercatinib is administered as a pharmaceutically acceptable salt. Selpercatinib is described, along with methods of its use as well as methods of its synthesis, in U.S. Pat. No. 10,112,942, incorporated herein by reference in its entirety for all purposes.

Osimertinib, having the chemical name of N-(2-{2-dimethylaminoethyl-methylamino}-4-methoxy-5-{[4-(1-methylindol-3 -yl)pyrimidin-2-yl]amino}phenyl)prop-2-enamide, is an epidermal growth factor receptor (EGFR) inhibitor having the chemical structure:

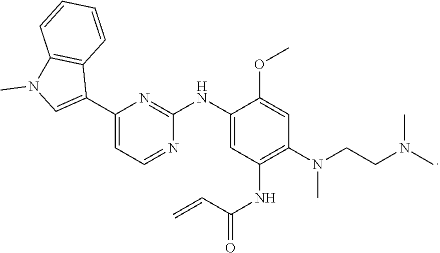

In typical embodiments, osimertinib is administered as a mesylate salt. However, other pharmaceutically acceptable salts of osimertinib are also contemplated to be used in the present disclosure. Osimertinib is described, along with methods of its use as well as methods of its synthesis, in U.S. Pat. No. 8,946,235, incorporated herein by reference in its entirety for all purposes.

The term “pharmaceutically acceptable salts”, as used herein, means salts of the active principal agents which are prepared with acids or bases that are tolerated by a biological system or tolerated by a patient or tolerated by a biological system and tolerated by a patient when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to, sodium, potassium, calcium, ammonium, organic amino, magnesium, lithium, strontium, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable addition salts include, but are not limited to: those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic and galactouronic acids and the like.

The term “RET-associated cancer” as used herein refers to cancers associated with or having a dysregulation of a RET gene, a RET kinase (also called herein a RET kinase protein), or expression or activity or level of any of the same. Non-limiting examples of RET-associated cancers are described herein.

The phrase “dysregulation of a RET gene, a RET kinase, or the expression or level or activity of any of the same” refers to a genetic mutation (e.g., a RET gene translocation that results in the expression of a fusion protein, a deletion in a RET gene that results in the expression of a RET protein that includes a deletion of at least one amino acid as compared to the wild-type RET protein, a mutation in a RET gene that results in the expression of a RET protein with one or more point mutations, or an alternative spliced version of a RET mRNA that results in a RET protein having a deletion of at least one amino acid in the RET protein as compared to the wild-type RET protein) or a RET gene amplification that results in overexpression of a RET protein or an autocrine activity resulting from the overexpression of a RET gene in a cell that results in a pathogenic increase in the activity of a kinase domain of a RET protein (e.g., a constitutively active kinase domain of a RET protein) in a cell. As another example, dysregulation of a RET gene, a RET protein, or expression or activity or level of any of the same, can be a mutation in a RET gene that encodes a RET protein that is constitutively active or has increased activity as compared to a protein encoded by a RET gene that does not include the mutation. For example, a dysregulation of a RET gene, a RET protein, or expression or activity or level of any of the same, can be the result of a gene or chromosome translocation which results in the expression of a fusion protein that contains a first portion of RET that includes a functional kinase domain, and a second portion of a partner protein (i.e., that is not RET). In some examples, dysregulation of a RET gene, a RET protein, or expression or activity or level of any of the same can be a result of a gene translocation of one RET gene with another non-RET gene. Non-limiting examples of fusion proteins are described in Table 2. Non-limiting examples of RET kinase protein point mutation, insertions, and deletions are described in Table 2. Additional examples of RET kinase protein mutations (e.g., point mutations) are RET inhibitor resistance mutations. Non-limiting examples of RET inhibitor resistance mutations are described in Tables 3 and 4.

The term “wildtype” or “wild-type” describes a nucleic acid (e.g., a RET gene or a RET mRNA) or protein (e.g., a RET protein) that is found in a patient that does not have a RET-associated cancer (and optionally also does not have an increased risk of developing a RET-associated cancer and/or is not suspected of having a RET-associated cancer), or is found in a cell or tissue from a patient that does not have a RET-associated cancer (and optionally also does not have an increased risk of developing a RET-associated cancer and/or is not suspected of having a RET-associated disease).

An exemplary sequence of human RET wild-type protein is an amino acid sequence of SEQ ID NO: 1 (UniProtKB P07949):

MAKATSGAAG LRLLLLLLLP LLGKVALGLY FSRDAYWEKL
YVDQAAGTPL LYVHALRDAP EEVPSFRLGQ HLYGTYRTRL
HENNWICIQE DTGLLYLNRS LDHSSWEKLS VRNRGFPLLT
VYLKVFLSPT SLREGECQWP GCARVYFSFF NTSFPACSSL
KPRELCFPET RPSFRIRENR PPGTFHQFRL LPVQFLCPNI
SVAYRLLEGE GLPFRCAPDS LEVSTRWALD REQREKYELV
AVCTVHAGAR EEWMVPFPV TVYDEDDSAP TFPAGVDTAS
AVVEFKRKED TVVATLRVFD ADVVPASGEL VRRYTSTLLP
GDTWAQQTFR VEHWPNETSV QANGSFVRAT VHDYRLVLNR
NLSISENRTM QLAVLVNDSD FQGPGAGVLL LHFNVSVLPV
SLHLPSTYSL SVSRRARRFA QIGKVCVENC QAFSGINVQY
KLHSSGANCS TLGVVTSAED TSGILFVNDT KALRRPKCAE
LHYMVVATDQ QTSRQAQAQL LVTVEGSYVA EEAGCPLSCA
VSKRRLECEE CGGLGSPTGR CEWRQGDGKG ITRNFSTCSP
STKTCPDGHC DVVETQDINI CPQDCLRGSI VGGHEPGEPR
GIKAGYGTCN CFPEEEKCFC EPEDIQDPLC DELCRTVIAA
AVLFSFIVSV LLSAFCIHCY HKFAHKPPIS SAEMTFRRPA
QAFPVSYSSS GARRPSLDSM ENQVSVDAFK ILEDPKWEFP
RKNLVLGKTL GEGEFGKVVK ATAFHLKGRA GYTTVAVKML
KENASPSELR DLLSEFNVLK QVNHPHVIKL YGACSQDGPL
LLIVEYAKYG SLRGFLRESR KVGPGYLGSG GSRNSSSLDH
PDERALTMGD LISFAWQISQ GMQYLAEMKL VHRDLAARNI
LVAEGRKMKI SDFGLSRDVY EEDSYVKRSQ GRIPVKWMAI
ESLFDHIYTT QSDVWSFGVL LWEIVTLGGN PYPGIPPERL
FNLLKTGHRM ERPDNCSEEM YRLMLQCWKQ EPDKRPVFAD
ISKDLEKMMV KRRDYLDLAA STPSDSLIYD DGLSEEETPL
VDCNNAPLPR ALPSTWIENK LYGMSDPNWP GESPVPLTRA
DGTNTGFPRY PNDSVYANWM LSPSAAKLMD TFDS

The oncogenic role of RET was firstly described in papillary thyroid carcinoma (PTC) (Grieco et al., Cell, 1990, 60, 557-63), which arises from follicular thyroid cells and is the most common thyroid malignancy. Approximately 20-30% of PTC harbor somatic chromosomal rearrangements (translocations or inversions) linking the promoter and the 5′ portions of constitutively expressed, unrelated genes to the RET tyrosine kinase domain (Greco et al., Q. J Nucl. Med Mal. Imaging, 2009, 53, 440-54), therefore driving its ectopic expression in thyroid cells. Fusion proteins generated by such rearrangements are termed “RET/PTC” proteins. For example, RET/PTC 1 is a fusion between CCDD6 and RET that is commonly found in papillary thyroid carcinomas. Similarly, both RET/PTC3 and RET/PTC4 are fusions of ELE1 and RET that are commonly found in papillary thyroid carcinomas, although the fusion events resulting RET/PTC3 and RET/PTC4 lead to different proteins with different molecular weights (see e.g., Fugazzola et al., Oncogene, 13(5): 1093-7, 1996). Some RET fusions associated with PTC are not referred to as “RET/PTC”, but instead are referred to as the fusion protein itself. For example, fusion between RET and both ELKS and PCM1 are found in PTCs, but the fusion proteins are referred to as ELKS-RET and PCM1-RET (see e.g., Romei and Elisei, Front. Endocrinol. (Lausanne), 3:54, doi: 10.3389/fendo.2012.00054, 2012). The role of RET-PTC rearrangements in the pathogenesis of PTC has been confirmed in transgenic mice (Santoro et al., Oncogene, 1996, 12, 1821-6). To date, a variety of fusion partners have been identified, from PTC and other cancer types, all providing a protein/protein interaction domain that induces ligand-independent RET dimerization and constitutive kinase activity (see, e.g., Table 1). Recently, a 10.6 Mb pericentric inversion in chromosome 10, where RET gene maps, has been identified in about 2% of lung adenocarcinoma patients, generating different variants of the chimeric gene KIFSB-RET (Ju et al., Genome Res., 2012, 22, 436-45; Kohno et al., 2012, Nature Med, 18, 375-7; Takeuchi et al., Nature Med, 2012, 18, 378-81; Lipson et al., 2012, Nature Med, 18, 382-4). The fusion transcripts are highly expressed and all the resulting chimeric proteins contain the N-terminal portion of the coiled-coil region of KIFSB, which mediates homodimerization, and the entire RET kinase domain. None of the RET positive patients harbor other known oncogenic alterations (such as EGFR or K-Ras mutation, ALK translocation), supporting the possibility that KIF5B-RET fusion could be a driver mutation of lung adenocarcinoma. The oncogenic potential of KIF5B-RET has been confirmed by transfection of the fusion gene into cultured cell lines. Similar to what has been observed with RET-PTC fusion proteins, KIF5B-RET is constitutively phosphorylated and induces NIH-3T3 transformation and IL-3 independent growth of BA-F3 cells. However, other RET fusion proteins have been identified in lung adenocarcinoma patients, such as the CCDC6-RET fusion protein, which has been found to play a key role in the proliferation of the human lung adenocarcinoma cell line LC-2/ad (Journal of Thoracic Oncology, 2012, 7(12): 1872-1876). RET inhibitors have been shown to be useful in treating lung cancers involving RET rearrangements (Drilon, A.E. et al. J Clin Oncol 33, 2015 (suppl; abstr 8007)). RET fusion proteins have also been identified in patients having colorectal cancer (Song Eun-Kee, et al. International Journal of Cancer, 2015, 136: 1967-1975).

Besides rearrangements of the RET sequence, gain of function point mutations of RET proto-oncogene are also driving oncogenic events, as shown in medullary thyroid carcinoma (MTC), which arises from parafollicular calcitonin-producing cells (de Groot, et al., Endocrine Rev., 2006, 27, 535-60; Wells and Santoro, Clin. Cancer Res., 2009, 15, 7119-7122). Around 25% of MTC are associated with multiple endocrine neoplasia type 2 (MEN2), a group of inherited cancer syndromes affecting neuroendocrine organs caused by germline activating point mutations of RET. In MEN2 subtypes (MEN2A, MEN2B and Familial MTC/FMTC) RET gene mutations have a strong phenotype-genotype correlation defining different MTC aggressiveness and clinical manifestations of the disease. In MEN2A syndrome mutations involve one of the six cysteine residues (mainly C634) located in the cysteine-rich extracellular region, leading to ligand independent homodimerization and constitutive RET activation. Patients develop MTC at a young age (onset at 5-25 years) and may also develop pheochromocytoma (50%) and hyperparathyroidism. MEN2B is mainly caused by M918T mutation, which is located in the kinase domain. This mutation constitutively activates RET in its monomeric state and alters substrate recognition by the kinase. MEN2B syndrome is characterized by an early onset (<1 year) and very aggressive form of MTC, pheochromocytoma (50% of patients) and ganglioneuromas. In FMTC the only disease manifestation is MTC, usually occurring at an adult age. Many different mutations have been detected, spanning the entire RET gene. The remaining 75% of MTC cases are sporadic and about 50% of them harbor RET somatic mutations: the most frequent mutation is M918T that, as in MEN2B, is associated with the most aggressive phenotype. Somatic point mutations of RET have also been described in other tumors such as colorectal cancer (Wood et al., Science, 2007, 318, 1108-13) and small cell lung carcinoma (Jpn. J Cancer Res., 1995, 86, 1127-30).

RET signaling components have been found to be expressed in primary breast tumors and to functionally interact with estrogen receptor-cc pathway in breast tumor cell lines (Boulay et al., Cancer Res. 2008, 68, 3743-51; Plaza-Menacho et al., Oncogene, 2010, 29, 4648-57), while RET expression and activation by GDNF family ligands could play an important role in perineural invasion by different types of cancer cells (Ito et al., Surgery, 2005, 138, 788-94; Gil et al., J. Natl. Cancer Inst., 2010, 102, 107-18; Iwahashi et al., Cancer, 2002, 94, 167-74).

RET is also expressed in 30-70% of invasive breast cancers, with expression being relatively more frequent in estrogen receptor-positive tumors (Plaza-Menacho, I., et al., Oncogene, 2010, 29, 4648-4657; Esseghir, S., et al., Cancer Res., 2007, 67, 11732-11741; Morandi, A, et al., Cancer Res., 2013, 73, 3783-3795; Gattelli, A, EMBO Mal. Med, 2013, 5, 1335-1350).

The identification of RET rearrangements has been reported in a subset of (patient derived xenograft) PDX established from colorectal cancer. Although the frequency of such events in colorectal cancer patients remains to be defined, these data suggest a role of RET as a target in this indication (Gozgit et al., AACR Annual Meeting 2014). Studies have shown that the RET promoter is frequently methylated in colorectal cancers, and heterozygous missense mutations, which are predicted to reduce RET expression, are identified in 5-10% of cases, which suggests that RET might have some features of a tumor suppressor in sporadic colon cancers (Luo, Y., et al., Oncogene, 2013, 32, 2037-2047; Sjoblom, T., et al., Science, 2006, 268-274; Cancer Genome Atlas Network, Nature, 2012, 487, 330-337).

An increasing number of tumor types are now being shown to express substantial levels of wild-type RET kinase that could have implications for tumor progression and spread. RET is expressed in 50-65% of pancreatic ductal carcinomas, and expression is more frequent in metastatic and higher grade tumors (Ito, Y, et al., Surgery, 2005, 138, 788-794; Zeng, Q., et al., J Int. Med Res. 2008, 36, 656-664).

In neoplasms of hematopoietic lineages, RET is expressed in acute myeloid leukemia (AML) with monocytic differentiation, as well as in CMML (Gattei, V. et al., Blood, 1997, 89, 2925-2937; Gattei, V., et al., Ann. Hematol, 1998, 77, 207-210; Camos, M., Cancer Res. 2006, 66, 6947-6954). Recent studies have identified rare chromosomal rearrangements that involve RET in patients with chronic myelomonocytic leukemia (CMML). CMML is frequently associated with rearrangements of several tyrosine kinases, which result in the expression of chimeric cytosolic oncoproteins that lead to activation of RAS pathways (Kohlmann, A, et al., J Clin. Oncol. 2010, 28, 2858-2865). In the case of RET, gene fusions that link RET with BCR (BCR-RET) or with fibroblast growth factor receptor 1 oncogene partner (FGFR1OP-RET) were transforming in early hematopoietic progenitor cells and could shift maturation of these cells towards monocytic paths, probably through the initiation of RET-mediated RAS signaling (Ballerini, P., et al., Leukemia, 2012, 26, 2384-2389).

RET expression has also been shown to occur in several other tumor types, including prostate cancer, small-cell lung carcinoma, melanoma, renal cell carcinoma, and head and neck tumors (Narita, N., et al., Oncogene, 2009, 28, 3058-3068; Mulligan, L. M., et al., Genes Chromosomes Cancer, 1998, 21, 326-332; Flavin, R., et al., Ural. Oneal., 2012, 30, 900-905; Dawson, D. M., J Natl Cancer Inst, 1998, 90, 519-523).

In neuroblastoma, RET expression and activation by GFLs has roles in tumor cell differentiation, potentially collaborating with other neurotrophic factor receptors to down regulate N-Myc, the expression of which is a marker of poor prognosis (Hofstra, R. M., W., et al., Hum. Genet. 1996, 97, 362-364; Petersen, S. and Bogenmann, E., Oncogene, 2004, 23, 213-225; Brodeur, G. M., Nature Ref Cancer, 2003, 3, 203-216).

In some embodiments, the RET-associated cancer is a hematological cancer. In some embodiments, the RET-associated cancer is a solid tumor. In some embodiments, the RET-associated cancer is lung cancer (e.g., small cell lung carcinoma or non-small cell lung carcinoma), thyroid cancer (e.g., papillary thyroid cancer, medullary thyroid cancer, differentiated thyroid cancer, recurrent thyroid cancer, or refractory differentiated thyroid cancer), thyroid adenoma, endocrine gland neoplasms, lung adenocarcinoma, bronchioles lung cell carcinoma, multiple endocrine neoplasia type 2A or 2B (MEN2A or MEN2Bmammary carcinoma, mammary neoplasm, colorectal cancer (e.g., metastatic colorectal cancer), papillary renal carcinoma, ganglioneuromatosis of the gastroenteric mucosa, inflammatory myofibroblastic tumor, or cervical cancer. In some embodiments, the RET-associated cancer is selected from the group consisting of: acute lymphoblastic leukemia (ALL), acute myeloid leukemia (AML), adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, chordoma, chronic lymphocytic leukemia (CLL), chronic myelogenous leukemia (CIVIL), chronic myeloproliferative neoplasms, neoplasms by site, neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, hepatocellular cancer, histiocytosis, Hodgkin's lymphoma, hypopharyneal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosystoma, pituitary cancer, plasma cell neoplasm, pleuropulmonary blastoma, pregnancy and breast cancer, primary central nervous system lymphoma, primary peritoneal cancer, prostate cancer, rectal cancer, colon cancer, colonic neoplasms, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, and Wilms' tumor.

In some embodiments, the RET-associated cancer is a hematological cancer selected from the group consisting of leukemias, lymphoma (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM). Additional examples of hematological cancers include myeloproliferative disorders (MPD) such as polycythemia vera (PV), essential thrombocytopenia (ET) and idiopathic primary myelofibrosis (IMF/IPF/PMF). In one embodiment, the RET-associated hematological cancer is AML or CMML.

In some embodiments, the RET-associated cancer is a solid tumor, examples of which include thyroid cancer (e.g., papillary thyroid carcinoma, medullary thyroid carcinoma), lung cancer (e.g., lung adenocarcinoma, small-cell lung carcinoma), pancreatic cancer, pancreatic ductal carcinoma, breast cancer, color cancer, colorectal cancer, prostate cancer, renal cell carcinoma, head and neck tumors, neuroblastoma, and melanoma.

In some embodiments, the RET-associated cancer is selected from the group consisting of lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

In some embodiments, the cancer is associated with dysregulation of a RET kinase, a RET gene, or the expression or activity or level of any of the same. Dysregulation of a RET kinase, a RET gene, or the expression or activity or level of any (e.g., one or more) of the same can contribute to tumorigenesis. For example, a dysregulation of a RET kinase, a RET gene, or expression or activity or level of any of the same can be a translocation, overexpression, activation, amplification, or mutation of a RET kinase, a RET gene, or a RET kinase domain. Translocation can include translocations involving the RET kinase domain, mutations can include mutations involving the RET ligand binding site, and amplification can be of a RET gene. Other dysregulations can include RET mRNA splice variants and RET autocrine/paracrine signaling, which can also contribute to tumorigenesis.

In some embodiments, the dysregulation of a RET gene, a RET kinase, or expression of activity of level of any of the same, includes overexpression of wild-type RET kinase (e.g., leading to autocrine activation). In some embodiments, the dysregulation of a RET gene, a RET kinase protein, or expression or activity or level of any of the same, includes overexpression, activation, amplification, or mutation in a chromosomal segment comprising the RET gene or a portion thereof, including, for example, the kinase domain portion, or a portion capable of exhibiting kinase activity.

In some embodiments, the dysregulation of a RET gene, a RET kinase protein, or expression or activity or level of any of the same, includes one or more chromosome translocations or inversions resulting in a RET gene fusion. In some embodiments, the dysregulation of a RET gene, a RET kinase protein, or expression or activity or level of any of the same, is a result of genetic translocations in which the expressed protein is a fusion protein containing residues from a non-RET partner protein and includes a minimum of a functional RET kinase domain.

Non-limiting examples of RET fusion proteins are provided in Table 1 below.

TABLE 1
Exemplary RET Fusion Partners and Cancers

| | Non-Limiting Exemplary RET-Associated |
|---|---|
| Fusion Partner | Cancer(s) |
| BCR | Chronic Myelomonocytic Leukemia |
| | (CMML) |
| CLIP1 | Adenocarcinoma |
| KIF5B | Non-Small Cell Lung Cancer, Ovarian |
| | Cancer, Spitzoid Neoplasms, Lung |
| | Adenocarcinoma, Adenosquamous |
| | Carcinomas |
| CCDC6 (also called PTC1, D10S170, or | Non-Small Cell Lung Cancer, Color Cancer, |
| H4) | Papillary Thyroid Cancer, |
| | Adenocarcinomas, Lung Adenocarcinoma, |
| | Metastatic Colorectal Cancer, |
| | Adenosquamous Carcinomas, Breast Cancer |
| PTC1ex9 (a CCDC6 rearrangement) | Metastatic papillary thyroid cancer |
| NCOA4 (also called PTC3, ELE1, and RFG) | Papillary Thyroid Cancer, Non-Small Cell |
| | Lung Cancer, Colon Cancer, Salivary Gland |
| | Cancer, Metastatic Colorectal Cancer, Lung |
| | Adenocarcinoma, Adenosquamous |
| | Carcinomas, Diffuse Sclerosing Variant of |
| | Papillary Thyroid Cancer, Breast Cancer, |
| | Actinic Cell Carcinoma, Mammary Analog |
| | Secretory Carcinoma |
| TRIM33 (also called PTC7 and RFG7) | Non-Small Cell Lung Cancer, Papillary |
| | Thyroid Cancer |
| ERC1 (also called ELKS) | Papillary Thyroid Cancer, Breast Cancer |
| FGFR1OP | CMML, Primary Myelofibrosis with |
| | secondary Acute Myeloid Leukemia |
| MBD1 (also known as PCM1) | Papillary Thyroid Cancer |
| RAB61P2 | Papillary Thyroid Cancer |
| PRKAR1A (also called PTC2) | Papillary Thyroid Cancer |
| TRIM24 (also called PTC6) | Papillary Thyroid Cancer |
| KTN1 (also called PTC8) | Papillary Thyroid Cancer |
| GOLGA5 (also called PTC5) | Papillary Thyroid Cancer, Spitzoid |
| | Neoplasms |
| HOOK3 | Papillary Thyroid Cancer |
| KIAA1468 (also called PTC9 and RFG9) | Papillary Thyroid Cancer, Lung |
| | Adenocarcinoma |
| TRIM27 (also called RFP) | Papillary Thyroid Cancer |
| AKAP13 | Papillary Thyroid Cancer |
| FKBP15 | Papillary Thyroid Cancer |
| SPECCIL | Papillary Thyroid Cancer, Thyroid Gland |
| | Carcinoma |
| TBL1XR1 | Papillary Thyroid Cancer, Thyroid Gland |
| | Carcinoma |
| CEP55 | Diffuse Gastric Cancer |
| CUX1 | Lung Adenocarcinoma |
| ACBD5 | Papillary Thyroid Carcinoma |
| MYH13 | Medullary Thyroid Carcinoma |
| Uncharacterized | Inflammatory Myofibroblastic Tumor |
| PIBF1 | Bronchiolus Lung Cell Carcinoma |
| KIAA1217 (also called SKT) | Papillary Thyroid Cancer, Lung |
| | Adenocarcinoma, NSCLC |
| MPRIP | NSCLC |
| HRH4 | Thyroid Cancer, Papillary Thyroid |
| | Carcinoma |
| Ria | Thyroid Cancer, Papillary Thyroid |
| | Carcinoma |
| RFG8 | Papillary Thyroid Carcinoma |
| FOXP4 | Lung Adenocarcinoma |
| MYH10 | Infantile Myofibromatosis |
| HTIF1 | Various |
| TIF1G | Various |
| H4L | Various |
| PTC4 (a NCO4/ELE1 rearrangement) | Papillary Thyroid Cancer |
| FRMD4A | NSCLC |
| SQSTM1 | Papillary Thyroid Carcinoma |
| AFAPIL2 | Papillary Thyroid Carcinoma |
| AFAP1 | NSCLC |
| PPFIBP2 | Papillary Thyroid Carcinoma |
| EML4 | Papillary Thyroid Carcinoma |
| PARD3 | NSCLC |
| RASGEF1A | Breast Cancer |
| TEL | In Vitro |
| RUFY1 | Colorectal Cancer |
| OLFM4 | Small-Bowel Cancer |
| UEVLD | Papillary Thyroid Carcinoma |
| DLG5 | Non-Anaplastic Thyroid NAT) Cancer |
| RRBP1 | Color Cancer |

In some embodiments, the RET gene fusion is selected from the group consisting of: BCR-RET, CLIP1-RET, KIF5B-RET, NCOA4-RET, TRIM33-RET, ERC1-RET, FGFR1OP-RET, RET-MBD1, RET-RAB61P2, RET-PRKAR1A, RET-TRIM24, RET-GOLGA5, HOOK3-RET, KTN1-RET, TRIM27-RET, AKAP13-RET, FKBP15-RET, SPECC1L-RET, TBL1XR1-RET, CEP55-RET, CUX1-RET, KIAA1468-RET, RFG8-RET, ACBD5-RET, PTC1ex9-RET, MYH13-RET, PIBF1-RET, KIAA1217-RET, MPRIP-RET, HRH4-RET, Ria-RET, RET-PTC4, FRMD4A-RET, HT1F1-RET, AFAP1-RET, RASGEF1A-RET, TEL-RET, RUFY1-RET, UEVLD-RET, DLG5-RET, FOXP4-RET, to TIF1G-RET, H4L-RET, OFLM4-RET, and RRBP1-RET.

In an embodiment, the RET gene fusion is selected from the group consisting of: RFG8-RET, HRH4-RET, Ria-RET, RET-PTC4, FRMD4A-RET, HT1F1-RET, AFAP1-RET, RASGEF1A-RET, TEL-RET, RUFY1-RET, UEVLD-RET, DLG5-RET, FOXP4-RET, TIF1G-RET, H4L-RET, OFLM4-RET, and RRBP1-RET.

In some embodiments, the RET gene fusion is CCDC6-RET. In some embodiments, the RET gene fusion is NCOA4-RET. In some embodiments, the RET gene fusion is KIF5B-RET. In some embodiments, the RET gene fusion is RUFY2-RET.

In some embodiments, the dysregulation of a RET gene, a RET kinase, or expression or activity or level of any of the same, includes one or more deletions (e.g., deletion of an amino acid at position 4), insertions, or point mutations in a RET kinase. In some embodiments, the dysregulation of a RET gene, a RET kinase, or expression or activity or level of any of the same, includes a deletion of one or more residues from the RET kinase, resulting in constitutive activity of the RET kinase domain.

In some embodiments, the dysregulation of a RET gene, a RET kinase, or expression or activity or level of any of the same, includes at least one point mutation in a RET gene that results in the production of a RET kinase that has one or more amino acid substitutions, insertions or deletions as compared to the wild-type RET kinase (see, for example, the point mutations listed in Table 2).

TABLE 2
Activating RET Kinase Protein Point Mutations/Insertions/Deletions
Exemplary RET Point Mutation

Amino acid position 2
Amino acid position 3
Amino acid position 4
Amino acid position 5
Amino acid position 6
Amino acid position 7
Amino acid position 8
Amino acid position 11
Amino acid position 12
Amino acid position 13
Amino acid position 20
Amino acid position 32 (e.g., S32L)
Amino acid position 34 (e.g., D34S)
Amino acid position 40 (e.g., L40P)
Amino acid position 56 (e.g., L56M)
Amino acid position 64 (e.g., P64L)
Amino acid position 67 (e.g., R67H)
Amino acid position 114 (e.g., R114H)
Amino acid position 136 (e.g., glutamic acid to stop codon)
Amino acid position 145 (e.g., V145G)
Amino acid position 180 (e.g., arginine to stop codon)
Amino acid position 200
Amino acid position 292 (e.g., V292M)
Amino acid position 294
Amino acid position 321 (e.g., G321R)
Amino acid position 330 (e.g., R330Q)
Amino acid position 338 (e.g., T338I)
Amino acid position 360 (e.g., R360W)
Amino acid position 373 (e.g., alanine to frameshift)
Amino acid position 393 (e.g., F393L)
Amino acid position 423 (e.g., G423R)
Amino acid position 432
Amino acid position 446 (e.g., G446R)
Δ Amino acid residues 505-506 (6-Base Pair In-Frame Germline Deletion in Exon 7)
Amino acid position 510 (e.g., A510V)
Amino acid position 511 (e.g., E511K)
Amino acid position 513 (e.g., G513D)
Amino acid position 514 (e.g., C515S, C515W)
Amino acid position 525 (e.g., R525W)
Amino acid position 531 (e.g., C531R or 9 base pair duplication)
Amino acid position 532 (e.g., duplication)
Amino acid position 533 (e.g., G533C, G533S)
Amino acid position 550 (e.g., G550E)
Amino acid position 591 (e.g., V591I)
Amino acid position 593 (e.g., G593E)
Amino acid position 595 (e.g., E595D and E595A)
Amino acid position 600 (e.g., R600Q)
Amino acid position 602 (e.g., I602V)
Amino acid position 603 (e.g., K603Q, K603E)
Amino acid position 606 (e.g., Y606C)
Amino acid position 609 (e.g., C609Y, C609S, C609G, C609R, C609F, C609W)
Amino acid position 611 (e.g., C611R, C611S, C611G, C611Y, C611F, C611W)
Amino acid position 616 (e.g., E615Q)
Amino acid position 618 (e.g. C618S, C618Y, C618R, C618Y, C618G, C618F, C618W)
Amino acid position 619
Amino acid position 620 (e.g., C620S, C620W, C620R, C620G, C620L, C620Y, C620F)
Amino acid position 623 (e.g., E623K)
Amino acid position 624 (e.g., D624N)
Amino acid position 630 (e.g., C630A, C630R, C630S, C630Y, C630F)
Amino acid position 631 (e.g., D631N, D631Y, D631A, D631G, D631V, D631E)
Amino acid position 632 (e.g., E632K, E632G)
Δ Amino acid residues 632-633 (6-Base Pair In-Frame Germline Deletion in Exon 11)
Amino acid position 633 (e.g., 9 base pair duplication)
Amino acid position 634 (e.g., C634W, C634Y, C634S, C634R, C634F, C634G, C634L,
C634A, or C634T, or an insertion ELCR, or a 12 base pair duplication)
Amino acid position 635 (e.g., R635G)
Amino acid position 636 (e.g., T636P, T636M)
Amino acid position 640 (e.g., A640G)
Amino acid position 641 (e.g., A641S, A641T)
Amino acid position 648 (e.g., V648I)
Amino acid position 649 (e.g., S649L)
Amino acid position 664 (e.g., A664D)
Amino acid position 665 (e.g., H665Q)
Amino acid position 666 (e.g., K666E, K666M, K666M, K666R)
Amino acid position 675 (silent nucleotide change)
Amino acid position 686 (e.g., S686N)
Amino acid position 689 (e.g., S689T)
Amino acid position 691 (e.g., G691S)
Amino acid position 694 (e.g., R694Q)
Amino acid position 700 (e.g., M700L)
Amino acid position 706 (e.g., V706M, V706A)
Amino acid position 713 splice variant (e.g., E713K)
Amino acid position 732 (e.g., E732K)
Amino acid position 736 (e.g., G736R)
Amino acid position 748 (e.g., G748C)
Amino acid position 750 (e.g., A750P)
Amino acid position 765 (e.g., S765P)
Amino acid position 766 (e.g., P766S, P766M)
Amino acid position 768 (e.g., E768Q, E768D0
Amino acid position 769
Amino acid position 770 (e.g., R770Q)
Amino acid position 771 (e.g., D771N)
Amino acid position 777 (e.g., N777S)
Amino acid position 778 (e.g., V778I)
Amino acid position 781 (e.g., Q781R)
Amino acid position 788
Amino acid position 790 (e.g., L790F)
Amino acid position 791 (e.g., Y791F, Y791N)
Amino acid position 802
Amino acid position 804 (e.g., V804L, V804M, V804E)
Amino acid position 805 (e.g., E805K)
Amino acid position804/805 (e.g., V804M/E805K)
Amino acid position 806 (e.g., Y806F, Y806S, Y806G, Y806C, Y806E, Y805H, Y806N)
Amino acid position 810 (e.g., G810R, G810S, G810A)
Amino acid position 818 (e.g., E818K)
Amino acid position 819 (e.g., S819I)
Amino acid position 823 (e.g., G823E)
Amino acid position 826 (e.g., Y826M, Y826S)
Amino acid position 833 (e.g., R833C)
Amino acid position 836
Amino acid position 841 (e.g., P841L)
Amino acid position 843 (e.g., E843D)
Amino acid position 844 (e.g., R844W, R844W, R844L)
Amino acid position 848 (e.g., M848T)
Amino acid position 852 (e.g., I852M)
Amino acid position 865 (e.g., L865V)
Amino acid position 870 (e.g., L870F)
Amino acid position 873 (e.g., R873W)
Amino acid position 876 (e.g., A876V)
Amino acid position 881 (e.g., L881V0
Amino acid position 882
Amino acid position 883 (e.g., A883F, A883S, A883T)
Amino acid position 884 (e.g., E884K)
Amino acid position 886 (e.g., R886W)
Amino acid position 891 (e.g., S891A)
Amino acid position 897 (e.g., R897Q)
Amino acid position 898 (e.g., D898V)
Amino acid position 900 (e.g., Y900F)
Amino acid position 901 (e.g., E901K)
Amino acid position 904 (e.g., S904F, S904C)
Amino acid position 905 (e.g., Y905F)
Amino acid position 907 (e.g., K907E, K907M)
Amino acid position 908 (e.g., (R908K)
Amino acid position 911 (e.g., G911D)
Amino acid position 912 (e.g., R912P, R912Q)
Amino acid position 918 (e.g., M918T, M918V, M918L)
Amino acid position 919 (e.g., A919V)
Amino acid position 921 (e.g., E912K)
Amino acid position 922 (e.g., S922P, S922Y)
Amino acid position 930 (e.g., T930M)
Amino acid position 961 (e.g., F961L)
Amino acid position 972 (e.g., R972G0
Amino acid position 981 (e.g., Y981F)
Amino acid position 982 (e.g., R982C)
Exemplary RET Point Mutation
Amino acid position 1009 (e.g., M1009V)
Amino acid position 1015 (e.g., Y1015F)
Amino acid position 1017 (e.g., D1017N)
Amino acid position 1041 (e.g., V1041G)
Amino acid position 1064 (e.g., M1064T)
Amino acid position 1096 (e.g., Y1096F)
RET + 3
(In Frame Deletion in Exons 6 and 11)
(3bp In-Frame Deletion in Exon 15)
Nucleotide position 2136 + 2 (e.g., 2136 + 27 > G)
(del632-636 ins6)
Amino acid positions 791 and 852 (e.g., Y791F + I852M)
Amino acid positions 634 and 852 (e.g., C634R + I852M)

In some embodiments, the one or more point mutations in the RET gene results in the translation of a RET protein having one or more amino acid substitutions at one or more of the following amino acid positions: 2, 3, 4, 5, 6, 7, 8, 11, 12, 13, 20, 32, 34, 40, 56, 64, 67, 114, 145, 292, 321, 330, 338, 360, 393, 423, 446, 510, 511, 513, 515, 525, 531, 532, 533, 550, 591, 593, 595, 602, 603, 606, 609, 611, 616, 618, 619, 620, 623, 624, 630, 631, 632, 634, 635, 636, 640, 641, 648, 649, 664, 665, 666, 675, 686, 689, 691, 694, 700, 706, 713, 732, 736, 748, 750, 765, 766, 768, 769, 770, 771, 777, 778, 781, 788, 790, 791, 804, 805, 806, 810, 818, 819, 823, 826, 833, 836, 841, 843, 844, 848, 852, 865, 870, 873, 876, 881, 883, 884, 886, 891, 897, 898, 900, 901, 904, 905, 907, 908, 911, 912, 918, 919, 921, 922, 930, 961, 972, 981, 982, 1009, 1015, 1017, 1041, 1064, or 1096.

In an embodiment, the one or more point mutations in the RET gene results in the translation of a RET protein having one or more of the following amino acid substitutions: S32L, D34S, L40P, L56M, P64L, R67H, RI 14H, V145G, V292M, G321R, R330Q, T3381, R360W, F393L, G423R, G446R, A510V, E511K, G513D, C515S, C515W, R525W, C531R, G533C, G533S, G550E, V5911, G593E, E595D, E595A, R600Q, 1602V, K603Q, K603E, Y606C, C609C, C609Y, C609S, C609G, C609R, C609F, C609W, C611R, C611S, C611G, C611Y, C611F, C611W, E616Q, C618S, C618Y, C618R, C618G, C618F, C618W, F619F, C620S, C620W, C620R, C620G, C620L, C620Y, C620F, E623K, D624N, C630A, C630R, C630S, C630Y, C630F, C630W, D631N, D631Y, D631A, D631G, D631V, D631E, E632K, E632G, C634W, C634Y, C634S, C634R, C634F, C634G, C634L, C634A, C634T, R635G, T636P, T636M, A640G, A641S, A641T, V6481, S649L, A664D, H665Q, K666E, K666M, K666N, K666R, T675T S686N, S689T, G691S, R694Q, M700L, V706M, V706A, E713K, E732K, G736R, G748C, A750P, S765P, P766S, P766M, E768Q, E768D, L769L, R770Q, D771N, N777S, V7781, Q781R, 17881, L790F, Y791F, Y791N, V804L, V804M, V804E, E805K, Y806E, Y806F, Y806S, Y806G, Y806C, Y806H, Y806N, Y806Y, G810R, G810S, G810A, E818K, S8191, G823E, Y826M, Y826S, R833C, S836S, P841L, P841P, E843D, R844W, R844Q, R844L, M848T, 1852M, L865V, L870F, R873W, A876V, L881V, A883F, A883S, A883T, E884K, R886W, S891A, S891S, R897Q, D898V, Y900F, E901K, S904F, S904S, S904C, Y905F, K907E, K907M, R908K, G911D, R912P, R912Q, M918T, M918V, M918L, A919V, E921K, S922P, S922Y, T930M, F961L, R972G, Y981F, R982C, M1009V, Y1015F, D1017N, V1041G, M1064T, or Y1096F.

In a further embodiment, the one or more point mutations in the RET gene results in the translation of a RET protein having one or more of the following amino acid substitutions: S32L, D34S, L40P, L56M, P64L, R67H, RI 14H, V145G, V292M, G321R, R330Q, T3381, R360W, F393L, G423R, G446R, A510V, E511K, G513D, C515S, C515W, R525W, C531R, G533C, G533S, G550E, V5911, G593E, E595D, E595A, R600Q, 1602V, K603Q, K603E, Y606C, C609C, C609Y, C609S, C609G, C609R, C609F, C609W, C611R, C611S, C611G, C611Y, C611F, C611W, E616Q, C618S, C618Y, C618R, C618G, C618F, C618W, F619F, C620S, C620W, C620R, C620G, C620L, C620Y, C620F, E623K, D624N, C630A, C630R, C630S, C630Y, C630F, C630W, D631N, D631Y, D631A, D631G, D631V, D631E, E632K, E632G, C634Y, C634S, C634R, C634F, C634G, C634L, C634A, C634T, R635G, T636P, T636M, A640G, A641S, A641T, V6481, S649L, A664D, H665Q, K666E, K666M, K666N, K666R, T675T S686N, S689T, G691S, R694Q, M700L, V706M, V706A, E713K, E732K, G736R, G748C, A750P, S765P, P766S, P766M, E768Q, E768D, L769L, R770Q, D771N, N777S, V7781, Q781R, 17881, L790F, Y791F, Y791N, V804E, E805K, Y806E, Y806F, Y806S, Y806G, Y806C, Y806H, Y806N, Y806Y, G810R, G810S, G810A, E818K, S8191, G823E, Y826M, Y826S, R833C, S836S, P841L, P841P, E843D, R844W, R844Q, R844L, M848T, 1852M, L865V, L870F, R873W, A876V, L881V, A883F, A883S, A883T, E884K, R886W, S891A, S891S, R897Q, D898V, Y900F, E901K, S904F, S904S, S904C, Y905F, K907E, K907M, R908K, G911D, R912P, R912Q, M918V, M918L, A919V, E921K, S922P, S922Y, T930M, F961L, R972G, Y981F, R982C, M1009V, Y1015F, D1017N, V1041G, M1064T, or Y1096F.

In some embodiments, the dysregulation of a RET gene, a RET kinase, or expression or activity or level of any of the same, includes a splice variation in a RET mRNA which results in an expressed protein that is an alternatively spliced variant of RET having at least one residue deleted (as compared to the wild-type RET kinase) resulting in constitutive activity of a RET kinase domain.

In some embodiments, the dysregulation of a RET gene, a RET kinase, or expression or activity or level of any of the same, includes at least one point mutation in a RET gene that results in production of a RET kinase that has one or more amino acid substitutions or insertions or deletion in a RET gene that has results in the production of a RET kinase that has one or more amino acids inserted or removed, as compared to the wild-type RET kinase. In some cases, the resulting RET kinase is more resistant to inhibition of its phosphotransferase activity by one or more RET kinase inhibitors, as compared to a to wildtype RET kinase or a RET kinase not including the same mutation. In such embodiments, a RET inhibitor resistance mutation can result in a RET kinase that has one or more of an increase Vₘₐₓ, a decreased Km for ATP, and an increased K_{D} for a RET kinase inhibitor, when in the presence of the RET kinase inhibitor, as compared to a wildtype RET kinase or a RET kinase not having the same mutation in the presence of the RET kinase inhibitor.

In other embodiments, the dysregulation of a RET gene, a RET kinase, or expression or activity or level of any of the same, includes at least one point mutation in a RET gene that results in the production of a RET kinase that has one or more amino acid substitutions as compared to the wild-type RET kinase, and which has increase resistance to selpercatinib, or a pharmaceutically acceptable salt thereof, as compared to a wildtype RET kinase or a RET kinase not including the same mutation. In such embodiments, a RET inhibitor resistance mutation can result in a RET kinase that has one or more of an increased Vₘₐₓ, a decreased Kₘ, and a decreased K_{D} in the presence of selpercatinib, or a pharmaceutically acceptable salt thereof, as compared to a wildtype RET kinase or a RET kinase not having the same mutation in the present of selpercatinib, or a pharmaceutically acceptable salt thereof.

Examples of RET inhibitor resistance mutation can, e.g., include point mutations, insertions, or deletions in and near the ATP binding site in the tertiary structure of RET kinase, including but not limited to the gatekeeper residue, P-loop residues, residues in or near the DFG motif, and ATP cleft solvent front amino acid residues. Additional examples of these types of mutations include changes in residues that may affect enzyme activity and/or drug binding including but are not limited to residues in the activation loop, residues near or interacting with the activation loop, residues contributing to active or inactive enzyme conformations, changes including mutations, deletions, and insertions in the loop proceeding the C-helix and in the C-helix. Specific residues or residue regions that may be changed (and are RET inhibitor resistance mutations) include but are not limited to those listed in Table 3 based on the human wildtype RET protein sequence (e.g., SEQ ID NO: 1).

TABLE 3
RET Inhibitor Resistance Mutations
Exemplary RET Inhibitor Resistance Mutations

Amino acid position 732 (e.g., E732K)
Amino acid position 788 (e.g., I788N)
Amino acid position 804 (e.g., V804M, V804L, V804E)
Amino acid position 804/805 (e.g., V804M/E805K)
Amino acid position 806 (e.g., Y806C, Y806E, Y806S, Y806H, Y806N)
Amino acid position 810 (e.g., G810A, G810R, G810S)
Amino acid position 865 (e.g., L865V)
Amino acid position 870 (e.g., L870F)

Additional examples of RET inhibitor resistance mutations are provided in Table 4 below.

TABLE 4
Additional Exemplary Amino Acid Positions
of RET Inhibitor Resistance Mutations

| RET | Exem- | |
|---|---|---|
| Amino | plary | |
| Acid and | Muta- | |
| Position | tion | Mechanistic Resistance Rationale |
| L730 | P | Steric hindrance and/or active conformational effect |
| G731 | V | Steric hindrance and/or active conformational effect |
| E732 | K | Steric hindrance and/or active conformational effect |
| G733 | V | Steric hindrance and/or active conformational effect |
| E734 | K | Steric hindrance and/or active conformational effect |
| L760 | M | Active conformational effect |
| K761 | E | Active conformational effect |
| E762 | K | Active conformational effect |
| N763 | D | Active conformational effect |
| A764 | V | Active conformational effect |
| S765 | N | Active conformational effect |
| P766 | A | Active conformational effect |
| S767 | C | Active conformational effect |
| E768 | K | Active conformational effect |
| L779 | M | Steric hindrance and/or active conformational effect |
| I788 | M | Steric hindrance and/or active conformational effect |
| M868 | R | Steric hindrance and/or active conformational effect |
| K869 | E | Steric hindrance and/or active conformational effect |
| L870 | Q | Steric hindrance and/or active conformational effect |
| V871 | M | Steric hindrance and/or active conformational effect |
| H872 | R | Steric hindrance and/or active conformational effect |
| R873 | P | Steric hindrance and/or active conformational effect |
| D874 | Y | Steric hindrance and/or active conformational effect |
| L881 | R | Steric hindrance and/or active conformational effect |
| L895 | M | Active conformational effect |
| S896 | N | Active conformational effect |
| R897 | C | Active conformational effect |
| D898 | Y | Active conformational effect |
| V899 | G | Active conformational effect |
| Y900 | D | Active conformational effect |
| E901 | K | Active conformational effect |
| E902 | K | Active conformational effect |
| D903 | Y | Active conformational effect |
| S904 | C | Active conformational effect |
| Y905 | D | Active conformational effect |
| V906 | M | Active conformational effect |
| K907 | E | Active conformational effect |
| R908 | P | Active conformational effect |
| S909 | C | Active conformational effect |
| Q910 | R | Active conformational effect |
| G911 | C | Active conformational effect |
| R912 | P | Active conformational effect |

In some embodiments, the one or more RET inhibitor resistance mutations results in the translation of a RET protein having one or more of the following amino acid substitutions: E732K, I788N, V804M, V804L, V804E, V804M/E805K, Y806C, Y806E, Y806S, Y806H, Y806N, G810A, G810R, G810S, L865V, or L870F.

In another embodiment, the one or more RET inhibitor resistance mutations results in the translation of a RET protein having one or more of the following amino acid substitutions: E732K, I788N, V804E, V804M/E805K, Y806C, Y806E, Y806S, Y806H, Y806N, G810A, G810R, G810S, L865V, or L870F.

In some embodiments, the patient has been identified or diagnosed as having a RET-associated cancer through the use of a regulatory agent-approved, e.g., FDA-approved, test or assay for identifying dysregulation of a RET gene, a RET kinase, or expression or activity or level of any of the same, in a patient or a biopsy sample from the patient or by performing any of the non-limiting examples of assays described herein. In some embodiments, the test or assay is provided as a kit.

In some embodiments, an assay is used to determine whether the patient has a dysregulation of a RET gene, or a RET kinase, or expression or activity or level of any of the same, using a sample from the patient. Such assays can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real time RT-PCR). As is well-known in the art, the assays are typically performed, e.g., with at least one labeled nucleic acid probe or at least one labeled antibody or antigen-binding fragment thereof. Assays can utilize other detection methods known in the art for detecting dysregulation of a RET gene, a RET kinase, or expression or activity or levels of any of the same. In some embodiments, the sample is a biological sample or a biopsy sample (e.g., a paraffin-embedded biopsy sample) from the patient.

In some embodiments, a method for treating a patient with an EGFR-associated cancer is provided comprising: administering a therapeutically effective amount of osimertinib, or a pharmaceutically acceptable salt thereof; determining whether a cancer cell in a sample obtained from the patient has at least one RET inhibitor resistance mutation; and administering a therapeutically effective amount of selpercatinib, or a pharmaceutically acceptable salt thereof, to the patient if the cancer cell obtained from the patient has at least one RET inhibitor resistance mutation; or administering one or more additional doses of osimertinib or its pharmaceutically acceptable salt if the cancer cell obtained from the patient does not have a RET inhibitor resistance mutation.

In some embodiments, a method for treating a patient with an EGFR-associated cancer is provided comprising: administering a therapeutically effective amount of osimertinib, or a pharmaceutically acceptable salt thereof; determining if the patient has one or more RET inhibitor resistance mutations; and if the patient is determined to have one or more RET inhibitor resistance mutations, then administering a therapeutically effective amount of selpercatinib, or a pharmaceutically acceptable salt thereof, to the patient.

When employed as pharmaceuticals, the compounds as used herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. In typical embodiments, the compounds as used in the methods described herein are administered as a pharmaceutical composition suitable for oral administration. In some embodiments, the dosage forms, such as tablets or capsules, are subdivided into suitable sized unit doses containing appropriate quantities of the active components, e.g., an effective amount to achieve the desired purpose.

The therapeutically effective dosage of selpercatinib, or a pharmaceutically acceptable salt thereof, and/or osimertinib, or a pharmaceutically acceptable salt thereof, will be typically determined by the health care practitioner depending on the condition, size, and age of the patient as well as the route of delivery.

In some embodiments, selpercatinib is administered as a free base. In some embodiments, selpercatinib is administered in a dosage form in an amount from 1 mg to 200 mg. For example, the dosage form may contain 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg selpercatinib.

In some embodiments, administering a therapeutically effective amount of selpercatinib comprises administering selpercatinib free base as a 40 mg dose twice a day. In some embodiments, administering a therapeutically effective amount of selpercatinib comprises administering selpercatinib free base as an 80 mg dose twice a day. In some embodiments, administering a therapeutically effective amount of selpercatinib comprises administering selpercatinib free base as a 120 mg dose twice a day. In some embodiments, administering a therapeutically effective amount of selpercatinib comprises administering selpercatinib free base as a 160 mg dose twice a day. In some embodiments, administering a therapeutically effective amount of selpercatinib comprises administering selpercatinib free base as a 100 mg dose once a day. In some embodiments, administering a therapeutically effective amount of selpercatinib comprises administering selpercatinib free base as a 160 mg dose once a day. In some embodiments, administering a therapeutically effective amount of selpercatinib comprises administering selpercatinib free base as a 240 mg dose once a day.

In some embodiments, osimertinib is administered as a mesylate salt, i.e., osimertinib mesylate. In some embodiments, osimertinib mesylate is administered in a dosage form in an amount from 1 mg to 200 mg. For example, the dosage form may contain 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg of osimertinib mesylate.

In some embodiments, administering a therapeutically effective amount of osimertinib comprises administering osimertinib mesylate salt as a 40 mg dose once a day. In some embodiment, administering osimertinib mesylate salt as an 80 mg dose once a day. In some embodiments, administering a therapeutically effective amount of osimertinib comprises administering osimertinib mesylate salt as a 40 mg dose twice a day. In some embodiments, administering a therapeutically effective amount of osimertinib comprises administering osimertinib mesylate salt as 80 mg dose twice a day.

In some embodiments, administering a therapeutically effective amount of selpercatinib and a therapeutically effective amount of osimertinib comprises administering osimertinib mesylate salt as a 40 mg dose once a day and selpercatinib as a 40 mg dose twice a day. In some embodiments, administering a therapeutically effective amount of selpercatinib and a therapeutically effective amount of osimertinib comprises administering osimertinib mesylate salt as a 40 mg dose once a day and selpercatinib as an 80 mg dose twice a day. In some embodiments, administering a therapeutically effective amount of selpercatinib and a therapeutically effective amount of osimertinib comprises administering osimertinib mesylate salt as a 40 mg dose once a day and selpercatinib as a 120 mg dose twice a day. In some embodiments, administering a therapeutically effective amount of selpercatinib and a therapeutically effective amount of osimertinib comprises administering osimertinib mesylate salt as a 40 mg dose once a day and selpercatinib as a 160 mg dose twice a day.

In some embodiments, administering a therapeutically effective amount of selpercatinib and a therapeutically effective amount of osimertinib comprises administering osimertinib mesylate salt as an 80 mg dose once a day and selpercatinib as a 40 mg dose twice a day. In some embodiments, administering a therapeutically effective amount of selpercatinib and a therapeutically effective amount of osimertinib comprises administering osimertinib mesylate salt as an 80 mg dose once a day and selpercatinib as an 80 mg dose twice a day. In some embodiments, administering a therapeutically effective amount of selpercatinib and a therapeutically effective amount of osimertinib comprises administering osimertinib mesylate salt as an 80 mg dose once a day and selpercatinib as a 120 mg dose twice a day. In some embodiments, administering a therapeutically effective amount of selpercatinib and a therapeutically effective amount of osimertinib comprises administering osimertinib mesylate salt as an 80 mg dose once a day and selpercatinib as a 160 mg dose twice a day.

In some embodiments, the combination of selpercatinib and osimertinib is administering for 1 week, 2 weeks, 3 weeks, 1 month, 2 months, 3 months, 4 months, 5 months, 6 months, 7 months, 8 months, 9 months, 10 months, 11 months 12 months, 13 months, 14 months, 15 months, 16 months, or more.

In some embodiments, the ratio of the daily dose of selpercatinib in mg to the daily dose of osimertinib in mg may be 1:1, 2:1, 3:1, 4:1, 5:1, 6:1, 7:1, 8:1 or more.

In some embodiments, selpercatinib, or its pharmaceutically acceptable salt, and/or osimertinib, or its pharmaceutically acceptable salt, either alone or in combination may be administered with at least one additional therapeutic agent. In some embodiments, the at least one additional therapeutic agent may comprise an anti-cancer agent, for example a chemotherapeutic agent.

A number of embodiments of the disclosure have been described. Nevertheless, it will be understood that various modifications may be made without departing from the spirit and scope of the invention. Accordingly, other embodiments are within the scope of the following claims.

The methods of the appended claims, in addition to the compositions used in said methods, are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of treating a patient with an EGFR-associated cancer and a RET-associated cancer, comprising administering to the patient a therapeutically effective amount of selpercatinib, or a pharmaceutically acceptable salt thereof, and a therapeutically effective amount of osimertinib, or a pharmaceutically acceptable salt thereof; wherein the RET-associated cancer is associated with a dysregulation of a RET gene, a RET kinase, or the expression or activity or level of any of the same; and wherein the dysregulation of a RET gene, a RET kinase, or the expression or activity or level of the same comprises a RET gene fusion.

2. The method of claim 1, wherein the RET gene fusion is selected from the group consisting of: RFG8-RET, HRH4-RET, Ria-RET, RET-PTC4, FRMD4A-RET, HT1F1-RET, AFAP1-RET, RAS GEF1A-RET, TEL-RET, RUFY1-RET, UEVLD-RET, DLG5-RET, FOXP4-RET, TIF1G-RET, H4L-RET, OFLM4-RET, and RRBP1-RET.

3. The method of claim 1, wherein the dysregulation of a RET gene, a RET kinase, or the expression of activity or level of the same comprises one or more point mutations in the RET gene.

4. The method of claim 3, wherein the one or more point mutations in the RET gene results in the translation of a RET protein having one or more amino acid substitutions at one or more of the following amino acid positions: 2, 3, 4, 5, 6, 7, 8, 11, 12, 13, 20, 32, 34, 40, 56, 64, 67, 114, 145, 292, 321, 330, 338, 360, 393, 423, 446, 510, 511, 513, 515, 525, 531, 532, 533, 550, 591, 593, 595, 602, 603, 606, 609, 611, 616, 618, 619, 620, 623, 624, 630, 631, 632, 634, 635, 636, 640, 641, 648, 649, 664, 665, 666, 675, 686, 689, 691, 694, 700, 706, 713, 732, 736, 748, 750, 765, 766, 768, 769, 770, 771, 777, 778, 781, 788, 790, 791, 804, 805, 806, 810, 818, 819, 823, 826, 833, 836, 841, 843, 844, 848, 852, 865, 870, 873, 876, 881, 883, 884, 886, 891, 897, 898, 900, 901, 904, 905, 907, 908, 911, 912, 918, 919, 921, 922, 930, 961, 972, 981, 982, 1009, 1015, 1017, 1041, 1064, or 1096.

5. The method of claim 4, wherein the one or more point mutations in the RET gene results in the translation of a RET protein having one or more of the following amino acid substitutions: S32L, D34S, L40P, L56M, P64L, R67H, RI 14H, V145G, V292M, G321R, R330Q, T3381, R360W, F393L, G423R, G446R, A510V, E511K, G513D, C515S, C515W, R525W, C531R, G533C, G533S, G550E, V5911, G593E, E595D, E595A, R600Q, 1602V, K603Q, K603E, Y606C, C609C, C609Y, C609S, C609G, C609R, C609F, C609W, C611R, C611S, C611G, C611Y, C611F, C611W, E616Q, C618S, C618Y, C618R, C618G, C618F, C618W, F619F, C620S, C620W, C620R, C620G, C620L, C620Y, C620F, E623K, D624N, C630A, C630R, C630S, C630Y, C630F, C630W, D631N, D631Y, D631A, D631G, D631V, D631E, E632K, E632G, C634Y, C634S, C634R, C634F, C634G, C634L, C634A, C634T, R635G, T636P, T636M, A640G, A641S, A641T, V6481, S649L, A664D, H665Q, K666E, K666M, K666N, K666R, T675T S686N, S689T, G691S, R694Q, M700L, V706M, V706A, E713K, E732K, G736R, G748C, A750P, S765P, P766S, P766M, E768Q, E768D, L769L, R770Q, D771N, N777S, V7781, Q781R, 17881, L790F, Y791F, Y791N, V804E, E805K, Y806E, Y806F, Y806S, Y806G, Y806C, Y806H, Y806N, Y806Y, G810R, G810S, G810A, E818K, 58191, G823E, Y826M, Y826S, R833C, S836S, P841L, P841P, E843D, R844W, R844Q, R844L, M848T, 1852M, L865V, L870F, R873W, A876V, L881V, A883F, A883S, A883T, E884K, R886W, S891A, S891S, R897Q, D898V, Y900F, E901K, S904F, S904S, S904C, Y905F, K907E, K907M, R908K, G911D, R912P, R912Q, M918V, M918L, A919V, E921K, S922P, S922Y, T930M, F961L, R972G, Y981F, R982C, M1009V, Y1015F, D1017N, V1041G, M1064T, or Y1096F.

6. The method of claim 1, wherein the dysregulation of a RET gene, a RET kinase, or the expression or activity or level of any of the same comprises one or more RET inhibitor resistance mutations.

7. The method of claim 6, wherein the one or more RET inhibitor resistance mutations results in the translation of a RET protein having one or more of the following amino acid substitutions: E732K, I788N, V804E, V804M/E805K, Y806C, Y806E, Y806S, Y806H, Y806N, G810A, G810R, G810S, L865V, or L870F.

8. A method of treating a patient with an EGFR-associated cancer comprising: administering a therapeutically effective amount of osimertinib, or a pharmaceutically acceptable salt thereof; determining if the patient has one or more RET inhibitor resistance mutations; and if the patient is determined to have one or more RET inhibitor resistance mutations, then administering a therapeutically effective amount of selpercatinib, or a pharmaceutically acceptable salt thereof, to the patient.

9. The method of claim 8, wherein the one or more RET inhibitor resistance mutations results in the translation of a RET protein having one or more of the following amino acid substitutions: E732K, I788N, V804E, V804M/E805K, Y806C, Y806E, Y806S, Y806H, Y806N, G810A, G810R, G810S, L865V, or L870F.

10. The method of claim 1, wherein osimertinib is administered as osimertinib mesylate.

11. The method of claim 10, wherein osimertinib mesylate is administered orally once a day.

12. The method of claim 10, wherein osimertinib mesylate is administered as a 40 mg dose.

13. The method of claim 10, wherein osimertinib mesylate is administered as an 80 mg dose.

14. The method of claim 1, wherein selpercatinib is administered as a free base.

15. The method of claim 1, wherein selpercatinib is administered orally twice a day.

16. The method of claim 15, wherein selpercatinib is administered as a 40 mg dose.

17. The method of claim 15, wherein selpercatinib is administered as an 80 mg dose.

18. The method of claim 15, wherein selpercatinib is administered as a 120 mg dose.

19. The method of claim 15, wherein selpercatinib is administered as a 160 mg dose.

20. The method of claim 1 wherein the RET-associated cancer is selected from the group consisting of: lung cancer, papillary thyroid cancer, medullary thyroid cancer, differentiated thyroid cancer, recurrent thyroid cancer, refractory differentiated thyroid cancer, multiple endocrine neoplasia type 2A or 2B, pheochomocytoma, parathyroid hyperplasia, breast cancer, colorectal cancer, papillary renal cell carcinoma, ganglioneuromatosis of the gastroenteric mucosa, and cervical cancer.
